(19) United States
(12) Patent Application Publication
(10) Pub. No.: US 2021/0045978 A1
(43) Pub. Date: Feb. 18, 2021

(54) O/W EMULSION COMPRISING PHOTOCHEMICALLY STABLE 4-(TERT-BUTYL)-4-METHOXYDIBENZOYLMETHANE IN OIL DROPLETS SMALLER THAN 8 MICROMETERS

(71) Applicant: BEIERSDORF AG, Hamburg (DE)

(72) Inventors: Tatjana SCHADE, Mildstedt/Rosendahl (DE); Heike VOLBRICH, Hamburg (DE)

(21) Appl. No.: 16/966,221

(22) PCT Filed: Jan. 22, 2019
(86) PCT No.: PCT/EP2019/051444

§ 371 Date: Jul. 30, 2020

(30) Foreign Application Priority Data
Feb. 2, 2018 (DE) 10 2018 201 594.5

(51) Int. Cl. A61K 8/06; A61K 8/35; A61Q 17/04

(52) U.S. Cl. CPC A61K 8/062; A61K 8/35; A61K 45/06; A61K 2800/30; A61K 2800/412; A61Q 17/04

(57) ABSTRACT

Cosmetic O/W emulsion containing 4-(tert.-butyl)-4′-methoxydibenzoylmethane, at least 90% of the oil droplets in the emulsion having a size (diameter) of less than 8 μm.

The present invention relates to a cosmetic oil-in-water emulsion with defined droplet size comprising 4-(tert-butyl)-4′-methoxydibenzoylmethane.

The trend away from pale complexion towards “healthy, athletic tanned skin” has continued for many years. To achieve this, people expose their skin to sunlight, since this causes pigment formation in the sense of melanin formation. However, the ultraviolet radiation of sunlight also has a harmful effect on skin. In addition to acute damage (sunburn), long-term damage such as an increased risk of developing skin cancer occurs on excessive irradiation with light from the UVB range (wavelength: 280-320 nm). Excessive exposure to UVB and UVA radiation (wavelength: 320-400 nm) additionally leads to a weakening of the elastic and collagen fibers in connective tissue. This leads to numerous phototoxic and photoallergic reactions and results in premature aging of the skin.

To protect the skin, a series of photoprotective filter substances have therefore been developed which may be used in cosmetic preparations. These UVA and UVB filters are in most industrialized countries collated in the form of positive lists such as Annex 7 of the Kosmetikverordnung [German Cosmetics Ordinance].

The plethora of commercially available sunscreens must not however obscure the fact that these prior art preparations have a number of disadvantages.

Cosmetic sunscreens typically comprise both UVA and UVB filters so that the preparation protects human skin from the entire spectrum of UV radiation. Most preparations comprise the compound 4-(tert-butyl)-4′-methoxydibenzoylmethane as UVA filter. However, cosmetic sunscreens comprising the UVA filter substance 4-(tert-butyl)-4′-methoxydibenzoylmethane have the problem that this compound is not particularly photostable and decomposes on exposure to UV radiation. Therefore, further stabilizers must be added to sunscreens comprising 4-(tert-butyl)-4′-methoxydibenzoylmethane as UV filter for the photostabilization thereof. According to the prior art, octocrylene is preferably used for the photostabilization of 4-(tert-butyl)-4′-methoxydibenzoylmethane.

The disadvantage of the prior art now consists in the fact that the use of octocrylene, despite approval by the licensing authorities, is not entirely uncontroversial and in evaluations of some consumer magazines (e.g. Oko-Test) results in “devaluations” in the rating of the product. The reason for this negative assessment is that some scientists suspect that this UV filter could possibly have hormonal effects. Even if no negative effects on humans have become known, despite decades of worldwide use of this UV filter in sunscreens, there is a desire among consumers to avoid preparations having such ingredients.

The object of the present invention was therefore to eliminate the disadvantages of the prior art and to develop a sunscreen in which the photochemical degradation of 4-(tert-butyl)-4′-methoxydibenzoylmethane is suppressed. Ideally, the object should be achieved without using octocrylene as solvent and stabilizer.

Surprisingly, the object is achieved by a cosmetic 01W emulsion comprising 4-(tert-butyl)-4′-methoxydibenzoylmethane, wherein at least 90% of the oil droplets in the emulsion have a size (diameter) of less than 8 μm.

Surprisingly, the object is achieved by a process for producing cosmetic O/W emulsions comprising 4-(tert-butyl)-4′-methoxydibenzoylmethane, the process comprising the following steps:

i) combining oil phase and water phase,

ii) pre-emulsification,

iii) adding temperature-sensitive ingredients such as perfumes,

iv) post-emulsification,

wherein the post-emulsification forms oil droplets with a droplet size in which at least 90% of the oil droplets have a size (diameter) of less than 8 μm.

Surprisingly, the object is also achieved by a process for the photostabilization of 4-(tert-butyl)-4′-methoxydibenzoylmethane in cosmetic 01W emulsions, wherein the 4-(tert-butyl)-4′-methoxydibenzoylmethane is incorporated in oil droplets, which to an extent of at least 90% have a size (diameter) of less than 8 μm.

Surprisingly, the object is achieved not least by the use of oil droplets with a droplet size (diameter) of less than 8 μm in cosmetic O/W emulsions for the photostabilization of 4-(tert-butyl)-4′-methoxydibenzoylmethane.

Surprisingly, it was also found that the emulsions according to the invention have a higher sun protection factor (SPF) than preparations with larger emulsion droplets.

Although the prior art certainly recognizes cosmetic O/W emulsions with 4-(tert-butyl)-4′-methoxydibenzoylmethane, it has so far been unrecognized that the droplet size of the emulsion has an influence on the photostability of 4-(tert-butyl)-4′-methoxydibenzoylmethane.

In the context of the present disclosure, the phrases “according to the invention”, “preparation according to the invention”, etc. refer in all cases to the preparations, processes and uses according to the invention, i.e. also to preparations in which the uses according to the invention are implemented and to preparations with which the processes according to the invention are implemented.

The droplet size of the oil droplets in the emulsion is determined according to the invention with the aid of laser diffraction measurement.

For this purpose, the Mastersizer 2000 measurement system from Malvern may be used. Here, the samples are dispersed in water and measured at room temperature (20°). The droplet size distribution is calculated using the model of Mie theory.
Refractive index of the particles: 1.469 Absorption 0.
Refractive index of dispersant water: 1.330.

Advantageous embodiments of the present invention are characterized in that the emulsion is homogenized using a rotor/stator device or a high-pressure homogenizer. These devices are preferably used for the post-emulsification.

In the context of the present invention, it is further advantageous when the preparation comprises 4-(tert-butyl)-4′-methoxydibenzoylmethane at a concentration of 1.0 to 5.0% by weight, based on the total weight of the emulsion.

In this case, the embodiments of the present invention that are advantageous according to the invention are characterized in that the preparation is free from 3-(4-methylbenzylidene)camphor, 2-hydroxy-4-methoxybenzophenone (INCI: Oxybenzone), ethylhexyl 2-cyano-3,3-diphenylacrylate (INCI: Octocrylene) and 2-ethylhexyl 4-methoxycinnamate (INCI Octyl Methoxycinnamate).

It is advantageous in the context of the present invention if the O/W emulsifier(s) are selected from the group of compounds comprising glyceryl stearate citrate, glyceryl stearate (self-emulsifying), stearic acid, stearate salts, polyglyceryl-3 methylglucose distearate, sodium cetearyl sulfate, potassium cetyl phosphate, polyglyceryl-10 stearate, sodium stearoyl glutamate.

In this case, the advantageous embodiments according to the invention are characterized in that the emulsifier(s) are used at a total concentration of 0.2 to 5% by weight, based on the total weight of the preparation.

In this case, the use of the emulsifier sodium stearoyl glutamate is preferred in accordance with the invention. For this emulsifier, the preferred use concentration according to the invention is from 0.2 to 0.5% by weight, based on the total weight of the preparation.

According to the invention, the emulsifier is preferably added to the water phase in the preparation of the emulsion.

It is also advantageous according to the invention if the preparation according to the invention is free from parabens, methylisothiazolinone, chloromethylisothiazolinone and DMDM hydantoin.

It is also advantageous according to the invention if the emulsion according to the invention is characterized in that the preparation contains one or more UV filters selected from the group of the compounds 2-phenylbenzimidazole-5-sulfonic acid and/or salts thereof; phenylene-1,4-bis(2-benzimidazyl)-3,3′-5,5′-tetrasulfonic acid salts; 1,4-di(2-oxo-10-sulfo-3-bornylidenemethyl)benzene and salts thereof; 4-(2-oxo-3-bornylidenemethyl)benzenesulfonic acid salts; 2-methyl-5-(2-oxo-3-bornylidenemethyl)sulfonic acid salts; 2,2′-methylenebis(6-(2H-benzotriazol-2-yl)-4-(1,1,3,3-tetramethylbutyl)phenol); 2-(2H-benzotriazol-2-yl)-4-methyl-6-[2-methyl-3-[1,3,3,3-tetramethyl-1-[(trimethylsilyl)oxy]disiloxanyl]propyl]phenol; ethylhexyl salicylate; terephthalidenedicamphorsulfonic acid; 2-ethylhexyl 4-(dimethylamino)benzoate; amyl 4-(dimethylamino)benzoate; di(2-ethylhexyl) 4-methoxybenzalmalonate; homomenthyl salicylate; 2-ethylhexyl 2-hydroxybenzoate; dimethicodiethylbenzalmalonate; 3-(4-(2,2-bisethoxycarbonylvinyl)phenoxy)propenyl)methoxysiloxane/dimethylsiloxane copolymer; hexyl 2-(4′-diethylamino-2′-hydroxybenzoyl)benzoate; dioctylbutylamidotriazone (INCI: Diethylhexyl Butamidotriazone); 2,4-bis[5-1-(dimethylpropyl)benzoxazol-2-yl-(4-phenyl)imino]-6-(2-ethylhexyl)imino-1,3,5-triazine with (CAS No. 288254-16-0); 2,4-bis{[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine); tris(2-ethylhexyl) 4,4′,4″-(1,3,5-triazine-2,4,6-triyltriimino)trisbenzoate (also: 2,4,6-tris[anilino-(p-carbo-2′-ethyl-1′-hexyloxy)]-1,3,5-triazine (INCI: Ethylhexyl Triazone); 2,4,6-tribiphenyl-4-yl-1,3,5-triazine; merocyanine; titanium dioxide; zinc oxide.

In this case, it is preferred in accordance with the invention to use 2,4-bis{[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine); tris(2-ethylhexyl) 4,4′,4″-(1,3,5-triazine-2,4,6-triyltriimino)trisbenzoate (also: 2,4,6-tris[anilino-(p-carbo-2′-ethyl-1′-hexyloxy)]-1,3,5-triazine (INCI: Ethylhexyl Triazone); ethylhexyl salicylate and also 2-phenylbenzimidazole-5-sulfonic acid and/or salts thereof.

Particularly preferred in accordance with the invention is the use of 2,4-bis{([4-(2-ethylhexyloxy)-2-hydroxy]phenyl}-6-(4-methoxyphenyl)-1,3,5-triazine (INCI: Bis-Ethylhexyloxyphenol Methoxyphenyl Triazine). This compound is advantageously used in accordance with the invention in an amount of 2 to 5% by weight, based on the total weight of the preparation.

The advantageous use concentrations according to the invention are for tris(2-ethylhexyl) 4,4′,4″-(1,3,5-triazine-2,4,6-triyltriimino)trisbenzoate (INCI: Ethylhexyl Triazone) 2 to 5% by weight, for 2-ethylhexyl 2-hydroxybenzoate (INCI: Ethylhexyl Salicylate) 3 to 5% by weight, for 2-phenylbenzimidazole-5-sulfonic acid and/or salts thereof 1 to 4% by weight, in each case based on the total weight of the preparation.

It is advantageous according to the invention if the preparation comprises ethanol, phenoxyethanol, 4-hydroxyacetophenone, 2-methylpropane-1,3-diol, 1,2-pentanediol, 1,2-hexanediol, 1,2-octanediol and/or 1,2-decanediol and/or ethylhexylglycerin.

Preference is given in accordance with the invention to the use of 4-hydroxyacetophenone, which is preferably used at a concentration of 0.01 to 2% by weight, based on the total weight of the preparation.

The cosmetic preparations according to the invention may also advantageously comprise fillers, although not mandatory, which further improve, for example, the sensory and cosmetic properties of the formulations and which generate or enhance, for example, a velvety or silky skin feel. Advantageous fillers in the context of the present invention are starch and starch derivatives (such as tapioca starch, distarch phosphate, aluminum or sodium starch octenyl succinate and the like), pigments, which have principally neither UV filter nor staining effects (e.g. boron nitride etc.) and/or Aerosils® (CAS No. 7631-86-9) and/or talc and/or polyethylene, nylon, silica dimethyl silylate.

It is preferred in accordance with the invention if the preparation according to the invention comprises silica dimethyl silylate.

Embodiments of the present invention that are advantageous in accordance with the invention are characterized in that the preparation contains one or more oils selected from the group of the compounds Butylene Glycol Dicaprylate/Dicaprate, Phenethyl Benzoate, C12-15 Alkyl Benzoate, Dibutyl Adipate, Diisopropyl Sebacate, Dicaprylyl Carbonate, Di-C12-13 Alkyl Tartrate, Butyloctyl Salicylate, Diethylhexyl Syringylidenemalonate, Hydrogenated Castor Oil Dimerate, Triheptanoin, C12-13 Alkyl Lactate, C16-17 Alkyl Benzoate, Propylheptyl Caprylate, Caprylic/Capric Triglyceride, Diethylhexyl 2,6-Naphthalate, Octyldodecanol, Caprylic/Capric Triglyceride, Ethylhexyl Cocoate.

In this case it is preferred in accordance with the invention if the preparation comprises dibutyl adipate, dicaprylyl carbonate and/or C12-C15 alkyl benzoate.

The water phase of the preparations according to the invention may advantageously comprise customary cosmetic auxiliaries such as alcohols, in particular those having a low number of carbon atoms, preferably ethanol and/or isopropanol or polyols having a low number of carbon atoms and also the ethers thereof, preferably propylene glycol, glycerin, electrolytes, self-tanning agents, and in particular one or more thickeners, which may advantageously be selected from the group silica, aluminum silicates, polysaccharides and derivatives thereof, for example hyaluronic acid, xanthan gum, hydroxypropyl methylcellulose, particularly advantageously from the group of polyacrylates, preferably a polyacrylate from the group of so-called Carbopols, for example Carbopols of types 980, 981, 1382, 2984, 5984, each individually or in combination). Further advantageous thickeners according to the invention are those with the INCI name Acrylates/C10-30 Alkyl Acrylate Crosspolymer (e.g. Pemulen TR 1, Pemulen TR 2, Carbopol 1328 from NOVEON) and Aristoflex AVC (INCI: Ammonium Acryloyldimethyltaurate/VP Copolymer).

It is preferred according to the invention here if the preparation comprises xanthan gum, crosslinked acrylate/C10-C30 alkyl acrylate polymer and/or vinyl pyrrolidone/hexadecene copolymer.

A glycerin content of at least 5% by weight, based on the total weight of the preparation, is particularly advantageous according to the invention.

Embodiments of the present invention that are advantageous in accordance with the invention are characterized in that the preparation contains one or more compounds selected from the group of the compounds alpha-lipoic acid, folic acid, phytoene, D-biotin, coenzyme Q10, alpha-glucosylrutin, carnitine, carnosine, natural and/or synthetic isoflavonoids, flavonoids, creatine, creatinine, taurine, β-alanine, panthenol, magnolol, honokiol, tocopheryl acetate, dihydroxyacetone, 8-hexadecene-1,16-dicarboxylic acid, glycerylglucose, (2-hydroxyethyl)urea, vitamin E and derivatives thereof, hyaluronic acid and/or salts thereof, and/or licochalcone A.

It is further advantageous in the context of the present invention if the preparation according to the invention contains one or more perfumes selected from the group of the compounds limonene, citral, linalool, alpha-isomethyl ionone, geraniol, citronellol, 2-isobutyl-4-hydroxy-4-methyltetrahydropyran, 2-tert-pentylcyclohexyl acetate, 3-methyl-5-phenyl-1-pentanol, 7-acetyl-1,1,3,4,4,6-hexamethyltetralin, adipic acid diesters, alpha-amylcinnamaldehyde, alpha-methyl ionone, amyl C butylphenylmethylpropionalcinnamal, amyl salicylate, amylcinnamyl alcohol, anise alcohol, benzoin, benzyl alcohol, benzyl benzoate, benzyl cinnamate, benzyl salicylate, bergamot oil, bitter orange oil, butylphenyl methylpropional, cardamom oil, cedrol, cinnamal, cinnamyl alcohol, citronellyl methylcrotonate, lemon oil, coumarin, diethyl succinate, ethyl linalool, eugenol, Evernia furfuracea extract, Evernia prunastri extract, farnesol, guaiac wood oil, hexylcinnamal, hexyl salicylate, hydroxycitronellal, lavender oil, limonene oil, linayl acetate, mandarin oil, menthyl PCA, methylheptenone, nutmeg oil, rosemary oil, sweet orange oil, terpineol, tonka bean oil, triethyl citrate and/or vanillin.

Advantageously in accordance with the invention, the preparation according to the invention contains film-forming agents. Film-forming agents in the context of the present invention are substances of different composition which are characterized by the following property: If a film-forming agent is dissolved in water or another suitable solvent and the solution is then applied to the skin, it forms a film after evaporation of the solvent which essentially serves to fix the light filter on the skin and thus to increase the water resistance of the product.

It is particularly advantageous to select the film-forming agents from the group of polymers based on polyvinylpyrrolidone (PVP).

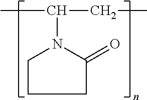

Particular preference is given to copolymers of polyvinylpyrrolidone, for example the PVP hexadecene copolymer and the PVP eicosene copolymer, which are obtainable under the trade names Antaron V216 and Antaron V220 from the GAF Chemicals Cooperation.

### COMPARATIVE EXPERIMENTS/WORKING EXAMPLES

The following formulations were prepared and the photostability of 4-(tert-butyl)-4′-methoxydibenzoylmethane was determined using the following method.

### Formulations:

| INCI | sample A | Phase | sample B | Phase | sample C | Phase | sample D | Phase |
|---|---|---|---|---|---|---|---|---|
| Isopropyl Palmitate | 7.00 | W | 7.00 | W | 7.00 | W | 7.00 | W |
| Aqua | 54.60 | O | 54.60 | O | 54.60 | O | 54.60 | O |
| Tocopheryl Acetate | 0.06 | O | 0.06 | O | 0.06 | O | 0.06 | O |
| Aqua + Trisodium EDTA | 1.00 | W | 1.00 | W | 1.00 | W | 1.00 | W |
| Dimethicone | 0.90 | O | 0.90 | O | 0.90 | O | 0.90 | O |
| Panthenol + Aqua | 1.40 | W | 1.40 | W | 1.40 | W | 1.40 | W |
| Butyl Methoxydibenzoylmethane | 2.38 | O | 2.38 | O | 2.38 | O | 2.38 | O |
| Phenylbenzimidazole Sulfonic Acid | 0.50 | W | 0.50 | W | 0.50 | W | 0.50 | W |
| VP/Hexadecene Copolymer | 0.50 | O | 0.50 | O | 0.50 | O | 0.50 | O |
| Behenyl Alcohol | 1.00 | O | 1.00 | O | 1.00 | O | 1.00 | O |
| Glycerin + Aqua | 7.50 | W | 7.50 | W | 7.50 | W | 7.50 | W |
| Aqua + Sodium Hydroxide | 0.11 | W | 0.11 | W | 0.11 | W | 0.11 | W |
| Alcohol Denat. + Aqua | 5.00 | A | 5.00 | A | 5.00 | A | 5.00 | A |
| Ethylhexyl Salicylate | 2.00 | O | 2.00 | O | 2.00 | O | 2.00 | O |
| Xanthan Gum | 0.40 | T | 0.40 | T | 0.40 | T | 0.40 | T |
| Silica Dimethyl Silylate | 1.00 | O | 1.00 | O | 1.00 | O | 1.00 | O |
| Stearyl Alcohol | 1.00 | O | 1.00 | O | 1.00 | O | 1.00 | O |
| Acrylates/C10-30 Alkyl Acrylate Crosspolymer | 0.10 | T | 0.10 | T | 0.10 | T | 0.10 | T |
| Ethylhexylglycerin | 0.20 | O | 0.20 | O | 0.20 | O | 0.20 | O |
| Bis-Ethylhexyloxyphenol Methoxyphenyl Triazine | 1.70 | O | 1.70 | O | 1.70 | O | 1.70 | O |
| Butylene Glycol Dicaprylate/Dicaprate | 3.00 | O | 3.00 | O | 3.00 | O | 3.00 | O |
| Tetrasodium Iminodisuccinate | 0.75 | W | 0.75 | W | 0.75 | W | 0.75 | W |
| Diethylamino Hydroxybenzoyl Hexyl Benzoate | 0.50 | O | 0.50 | O | 0.50 | O | 0.50 | O |
| Sodium Stearoyl Glutamate | 0.30 | O | 0.30 | O | 0.30 | W | 0.30 | W |
| C12-15 Alkyl Benzoate | 6.00 | O | 6.00 | O | 6.00 | O | 6.00 | O |
| Hydroxyacetophenone | 0.20 | W | 0.20 | W | 0.20 | W | 0.20 | W |
| Cellulose Gum | 0.50 | T | 0.50 | T | 0.50 | T | 0.50 | T |
| Parfum | 0.40 | A | 0.40 | A | 0.40 | A | 0.40 | A |
| | 100.00 | | 100.00 | | 100.00 | | 100.00 | |

W = Water phase
O = Oil phase
A = Alcohol phase
T = Thickener

### Preparation:

The phases W and O are heated separately to 80-85° C. After adding phase T to phase O, the two phases O and W are combined in a mixer. After combining, the batches are homogenized under reduced pressure at 75° C. and cooled to 30-40° C. After addition of phase A, the mixture is stirred for 10 minutes. At 30° C., energy is again introduced under reduced pressure by homogenization and the mixture then cooled to room temperature.

Energy was introduced into the mixtures according to the following scheme:

| Manufacturing | sample A | sample B | sample C | sample D |
|---|---|---|---|---|
| Type of mixer | Becomix RW 125 | Becomix RW 125 | Becomix RW 125 | Conti system |
| Energy input 75° C. | 3 min 14 m/s | 3 min 14 m/s | 3 min 14 m/s | |
| Energy input 30° C. | 5 min 25 m/s | 15 min 25 m/s | 15 min 25 m/s | |
| Energy input Mixer 1 | | | | 20 |
| 80° C. [m/s] [1/min] | | | | |
| Energy input Mixer 2 | | | | 3200 |
| 75° C. [m/s] [1/min] | | | | |
| Energy input Mixer 1 | | | | 10 |
| 30° C. [m/s] [1/min] | | | | |
| Energy input BTO | 1 | 3 | 3 | 3 |
| (Batch Turn Over) | | | | |

### Determination of the Photostability

The following formulations were prepared and the photostability of 4-(tert-butyl)-4′-methoxydibenzoylmethane was determined.

### Method(s) Used:

Processing and analysis based on the COLIPA method “In Vitro Determination of Photostability of Suncare Products” from October 1999, but without pre-irradiation. Dose ca. 250 kJ/m²

### Determination in Triplicate

Apparatus: Atlas Suntest CPS+with standard filter and water cooling Device No.134653
Dose 290-400 nm: ca. 250 kJ/m² (58 min, 11s at 765 W/m² lamp power)
Application amount: 2 mg/cm²
Eusoles 9020 from Merck was used as standard substance (# 5844J093)

### Determination of the Droplet Size:

Laser diffraction measurement (Mastersizer 2000 measuring system from Malvern) The samples were dispersed in water and measured at room temperature (20°). The droplet size distribution was calculated using the model of Mie theory.
Refractive index of the particles: 1.469 Absorption 0.
Refractive index of dispersant water: 1.330.

### Results:

| | sam- | sam- | sam- | sam- |
|---|---|---|---|---|
| | ple A | ple B | ple C | ple D |
| Droplet size 90% (μm) | 10.3 | 8.5 | 7.9 | 5.5 |
| SPF | 34.7 | 30.5 | 43.3 | 48.5 |
| Recovery BMDM after | 71 | 72 | 75 | 76 |
| Irradiation (250 kJ/m²) [%] | | | | |

1.-14. (canceled)

15. A cosmetic O/W emulsion, wherein the emulsion comprises 4-(tert-butyl)-4′-methoxydibenzoylmethane and at least 90% of oil droplets in the emulsion have a size (diameter) of less than 8 μm.

16. The emulsion of claim 15, wherein at least 90% of the oil droplets in the emulsion have a size (diameter) of above 350 nm.

17. The emulsion of claim 15, wherein the emulsion has been homogenized using a rotor/stator device or a high-pressure homogenizer.

18. The emulsion of claim 15, wherein the emulsion comprises from 1.0% to 5.0% of 4-(tert-butyl)-4′-methoxydibenzoylmethane, based on a total weight of the emulsion.

19. The emulsion of claim 15, wherein the emulsion is free from 3-(4-methylbenzylidene)camphor, 2-hydroxy-4-methoxybenzophenone (INCI: Oxybenzone), ethylhexyl 2-cyano-3,3-diphenylacrylate (INCI: Octocrylene) and 2-ethylhexyl 4-methoxycinnamate (INCI Octyl Methoxycinnamate).

20. The emulsion of claim 15, wherein the emulsion comprises one or more emulsifiers selected from glyceryl stearate citrate, glyceryl stearate (self-emulsifying), stearic acid, stearate salts, polyglyceryl-3 methylglucose distearate, sodium cetearyl sulfate, potassium cetyl phosphate, polyglyceryl-10 stearate, sodium stearoyl glutamate.

21. The emulsion of claim 20, wherein the emulsion comprises sodium stearoyl glutamate.

22. The emulsion of claim 21, wherein the emulsion comprises from 0.2% to 0.5% by weight of sodium stearoyl glutamate, based on a total weight of the preparation.

23. The emulsion of claim 15, wherein the emulsion is free from parabens, methyl-isothiazolinone, chloromethylisothiazolinone and DMDM hydantoin.

24. The emulsion of claim 15, wherein the emulsion further comprises one or more UV filters selected from 2-phenylbenzimidazole-5-sulfonic acid and/or salts thereof; phenylene-1,4-bis(2-benzimidazyl)-3,3′-5,5′-tetrasulfonic acid salts; 1,4-di(2-oxo-10-sulfo-3-bornylidenemethyl)benzene and salts thereof; 4-(2-oxo-3-bornylidenemethyl)benzenesulfonic acid salts; 2-methyl-5-(2-oxo-3-bornylidenemethyl)sulfonic acid salts; 2,2′-methylenebis(6-(2H-benzotriazol-2-yl)-4-(1,1,3,3-tetramethylbutyl)phenol); 2-(2H-benzotriazol-2-yl)-4-methyl-6-[2-methyl-3-[1,3,3,3-tetramethyl-1-[(trimethylsilyl)oxy]disiloxanyl]propyl]phenol; ethylhexyl salicylate; terephthalidenedicamphorsulfonic acid; 2-ethylhexyl 4-(dimethylamino)benzoate; amyl 4-(dimethylamino)benzoate; di(2-ethylhexyl) 4-methoxybenzalmalonate; homomenthyl salicylate; 2-ethylhexyl 2-hydroxybenzoate; dimethicodiethylbenzalmalonate; 3-(4-(2,2-bisethoxycarbonylvinyl)phenoxy)propenyl)methoxysiloxane/dimethylsiloxane copolymer; hexyl 2-(4′-diethylamino-2′-hydroxybenzoyl)benzoate dioctylbutylamidotriazone (INCI: Diethylhexyl Butamidotriazone); 2,4-bis[5-1(dimethylpropyl)benzoxazol-2-yl-(4-phenyl)imino]-6-(2-ethylhexyl)imino-1,3,5-triazine with (CAS No. 288254-16-0); 2,4-bis{[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine); tris(2-ethylhexyl) 4,4′,4″-(1,3,5-triazine-2,4,6-triyltriimino)trisbenzoate (also: 2,4,6-tris[anilino-(p-carbo-2′-ethyl-1′-hexyloxy)]-1,3,5-triazine (INCI: Ethylhexyl Triazone); 2,4,6-tribiphenyl-4-yl-1,3,5-triazine; merocyanine; titanium dioxide; zinc oxide.

25. The emulsion of claim 24, wherein the emulsion comprises 2,4-bis{[4-(2-ethylhexyloxy)-2-hydroxy]phenyl}-6-(4-methoxyphenyl)-1,3,5-triazine.

26. The emulsion of claim 25, wherein the emulsion comprises from 2% to 5% by weight of 2,4-bis{[4-(2-ethylhexyloxy)-2-hydroxy]phenyl}-6-(4-methoxyphenyl)-1,3,5-triazine, based on a total weight of the emulsion.

27. The emulsion of claim 15, wherein the emulsion comprises from 0.2% to 5% by weight of one or more emulsifiers, based on a total weight of the emulsion.

28. The emulsion of claim 15, wherein the emulsion further comprises from 2% to 5% by weight of tris(2-ethylhexyl) 4,4′,4″-(1,3,5-triazine-2,4,6-triyltriimino)trisbenzoate (INCI: Ethylhexyl Triazone) and/or from 3% to 5% by weight of 2-ethylhexyl 2-hydroxybenzoate (INCI: Ethylhexyl Salicylate) and/or from 1% to 4% by weight of 2-phenylbenzimidazole-5-sulfonic acid and/or salts thereof, in each case based on a total weight of the emulsion.

29. The emulsion of claim 15, wherein the emulsion further comprises one or more of ethanol, phenoxyethanol, 4-hydroxyacetophenone, 2-methylpropane-1,3-diol, 1,2-pentanediol, 1,2-hexanediol, 1,2-octanediol, 1,2-decanediol, ethylhexylglycerin.

30. The emulsion of claim 29, wherein the emulsion comprises 4-hydroxy-acetophenone.

31. The emulsion of claim 30, wherein the emulsion comprises from 0.01% to 2% by weight of 4-hydroxyacetophenone, based on a total weight of the emulsion.

32. A process for producing a cosmetic O/W emulsion comprising 4-(tert-butyl)-4′-methoxydibenzoylmethane, wherein the process comprises:
(i) combining oil phase and water phase,
(ii) pre-emulsification,
(iii) adding temperature-sensitive ingredients,
(iv) post-emulsification,
and wherein the post-emulsification forms oil droplets of which at least 90% have a size (diameter) of less than 8 μm.

33. The process of claim 32, wherein one or more emulsifiers are added to the water phase of (i).

34. A process for the photostabilization of 4-(tert-butyl)-4′-methoxydibenzoyl-methane in a cosmetic O/W emulsion, wherein the process comprises incorporating the 4-(tert-butyl)-4′-methoxydibenzoylmethane into oil droplets of which at least 90% have a size (diameter) of less than 8 μm.
